(19) United States
(12) Patent Application Publication
(10) Pub. No.: US 2005/0208149 A1
(43) Pub. Date: Sep. 22, 2005

(54) Use of arsenic-containing pharmaceutical composition in combination with radiation therapy for cancer treatment

(75) Inventors: Yuen-Liang Lai, Taipei (TW); Yu-Jen Chen, Taipei (TW); Yu-Fang Hu, Taipei (TW); Chi-Liang Kan, Taipei (TW); Kuang-Chun Chiu, Taipei (TW)

Correspondence Address: COHEN, PONTANI, LIEBERMAN & PAVANE;Suite 1210, 551 Fifth Avenue, New York, NY, 10176, US

(73) Assignee: TTY Biopharm Limited Company

(21) Appl. No.: 10/803,666

(22) Filed: Mar. 18, 2004

(51) Int. Cl. A61K 33/36; A61N 5/00

(52) U.S. Cl. 424/623; 600/1

(57) ABSTRACT

The present invention discloses the combination use of arsenic-containing pharmaceutical composition and radiation therapy on cutaneous metastases.

### BACKGROUND OF THE INVENTION

1. Field of the Invention

This invention relates to use of an arsenic-containing pharmaceutical composition in combination with radiation for the treatment of tumors/cancers. Particularly, the arsenic-containing compound selected from a group consisting of AS2O₃, AS₂S₃, As₂S₂ and a combination thereof is formulated into a pharmaceutical composition and used in combination with radiation for the treatment of cutaneous metastatic cancers.

2. Description of the Related Art

Tumors/cancers have always been a threat to the health of human beings. For many years, the medicinal field has endeavored to research and develop effective medicines for the treatment of tumors/cancers. However, up to the present, there is no medicine that is effective in the clinical treatment of tumors/cancers, for example, in the treatment of cutaneous metastatic cancers. The most common sources of cutaneous metastatic cancers are cancers of breast, colon, ovaries, lungs, head-and-neck and oral cavity. [Cheng C F, et al., J Intern Med Taiwan 2003; 14:31-36.]

Early in the 20th century, arsenic compounds were used to treat chronic myelogenous leukemia and malignant lymphoma. Recently, arsenic trioxide (AS2O₃) has been used in the treatment of de novo and refractory acute promyelocytic leukemia (APL) patients, with high rates of response and hematological/molecular remission achieved [Soignet S L, et. al., N Engl J Med 1998; 339:1341-1348.]. Clinical response has also been obtained for patients with human T cell lymphotropic virus type I associated adult T cell leukemia/lymphoma [Bazarbachi A, & Hermine O., Virus Res 2001; 78:79-92.]. Since 1990, arsenic-containing compounds (arsenicals) have provided another direction for research and development in the treatment of tumors/cancers. Further, As₂O₃ inhibits cell growth and induces apoptosis in certain types of cancer, including APL [Shao W, et. al., J Natl Cancer I 1998; 90:124-133.], hepatoma [Siu K P, et. al., Life Sci. 2002; 71:275-285.] as well as pancreatic [Li X, et. al., Anticancer Res 2002; 22:2205-2213.] and gastric [Jiang X H, et. al., Int J Cancer 2001; 91:173-179.] carcinomas. It was reported that, other than arsenic trioxide, arsenic-containing substances such as “Composite Indigo Naturalis Tablets” containing arsenic sulfide (As₂S₂) and pure tetraarsenic tetraslfide (As₄S₄) can achieve complete remission rates of 98% and 84.9%, respectively. [Wang Z. Y., Cancer Chemother Pharmacol (2001), 48 (suppl 1): S72-S76].

In 2001, the National Institutes of Health (NIH) of the United States proceeded with clinical trials of arsenic trioxide in hematologic and solid tumors, and indicated in their reports that arsenic trioxide can inhibit growth of many cancer cell lines, and promote apoptosis in the cancer cell lines. The clinical trials of arsenic trioxide conducted in connection with hematologic malignancies include, in addition to acute promyelocytic leukemia, acute myeloid leukemia (AML), acute lymphocytic leukemia, chronic myelogenous leukemia (CML), non-Hodgkin's lymphoma, Hodgkin's lymphoma, chronic lymphocytic leukemia, myelodysplastic syndrome and multiple myeloma. The clinical trials of arsenic trioxide conducted in connection with solid tumors include prostate cancer, cervical cancer and bladder cancer. [Murgo A. J., The Oncologist (2001), 6 (suppl 2):22-28].

In September 2000, the Food and Drug Administration (FDA) of the United States approved arsenic trioxide as an orphan drug for treating acute promyelocytic leukemia. The Department of Health (DOH) of Taiwan, R.O.C., approved marketing of an arsenic trioxide-containing pharmaceutical preparation (“ASADIN Injection”; license no. 000005) by TTY Biopharm Co., Ltd. (Taiwan), in January 2002.

In the treatment of tumors/cancers, radiation has been used by the medical field in the diagnosis and treatment of diseases, particularly in the treatment of cancers (such as skin cancers and nasopharyngeal cancers) since the discovery of the radioactive Ra element (²²⁶Ra) by the Curies in 1898. With the development in the research of the radionuclide science after World War II, scientists have gained a better understanding of the effects of radiation on living creatures, which has further improved methods and techniques of radiation therapy, increased the rate of survival, and prolonged lifespan, while reducing the side effects of radiation on normal tissues.

Chun et al., have demonstrated that As₂O₃ can sensitize human cervical cancer cells to ionizing radiation, both in vitro and in vivo. [Chun Y J, et. al., FEBS Lett. 2002; 519:195-200.]. In combination with ionizing radiation, As₂O₃ pre-treatment has a synergistic effect with respect to decreased clonogenic survival and regression of established human cervical tumor xenografts [Chun Y J, et. al., FEBS Lett 2002; 519:195-200.]. At the time of writing, however, the combined effect of As₂O₃ and radiation on clinical cancer patients had not been reported. Further, no discussion in Chun et al. was made regarding whether the treatment of cervical cancer can be extended to cutaneous metastatic cancers.

In the previous study for cutaneous metastatic breast cancer, it was demonstrated that topical As2O₃ improved local tumor control and decreased wound secretion without significant systemic or local adverse effects [Cheng C F, et al., J Intern Med Taiwan 2003; 14:31-36.].

However, treatment of cutaneous metastatic cancer with radiation therapy or chemotherapy alone have shown to have generally disappointing results.

In this present invention, the inventors examined the efficacy and safety of radiation in combination with As₂O₃ pre-treatment for palliation of superficial malignant lesions in breast cancer patients. For efficacy assessment, the diameters of involved skin metastasis area, pain score and daily needed frequency of the changing dressing (CD) were recorded. The dermatological, gastrointestinal, hematological, renal and hepatic toxicities of the treatment were also monitored for safety consideration.

All literature and patents mentioned hereinabove, as well as the literature cited herein, are incorporated herein by reference in their entirety.

### SUMMARY OF THE INVENTION

Accordingly, it is an aspect of the invention to provide a pharmaceutical composition and its use in combination with radiation for cancer therapy.

In one aspect, the invention provides a method for treating a human patient having a cutaneuous metastatic cancer comprising (a) administering topically an arsenic-containing pharmaceutical composition to a site of the cutaneuous metastatic cancer of human patient, the arsenic-containing pharmaceutical composition including a therapeutically effective amount of an arsenic-containing compound and a pharmaceutically acceptable carrier; and (b) applying transcutaneously an electron beam to the site so as to permit delivery of the arsenic-containing pharmaceutical composition to penetrate into cells of the cutaneuous metastatic cancer.

In other aspect, the invention provides an arsenic-containing pharmaceutical composition for treating cutanuoes metastatic cancers, wherein the pharmaceutical composition includes a therapeutically effective amount of an arsenic-containing compound which is selected from the group consisting of As₂O₃, As2S3, As2S2 and a combination thereof.

These and other aspects and features of the invention will be more fully appreciated when the following detailed description of the invention is read in conjunction with the accompanying drawings.

### BRIEF DESCRIPTION OF THE DRAWING

In the drawing:

FIG. 1 shows Skin-infiltrating tumor of a breast cancer patient (patient 1): (A) before combined treatment and (B) 3 weeks after combined treatment.

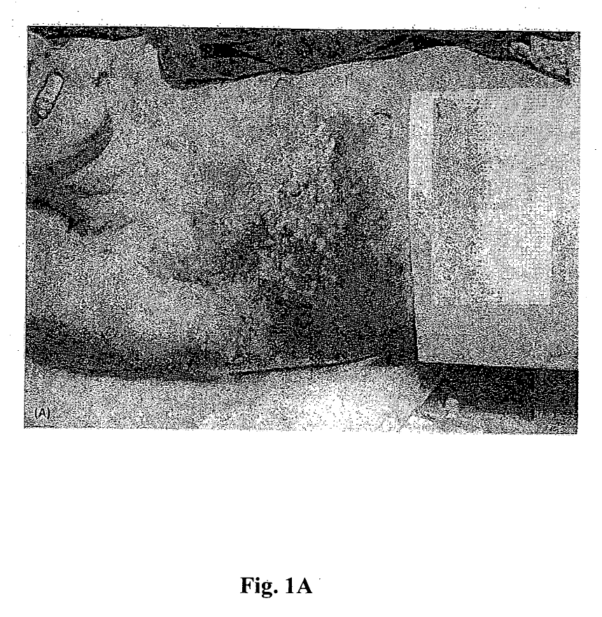

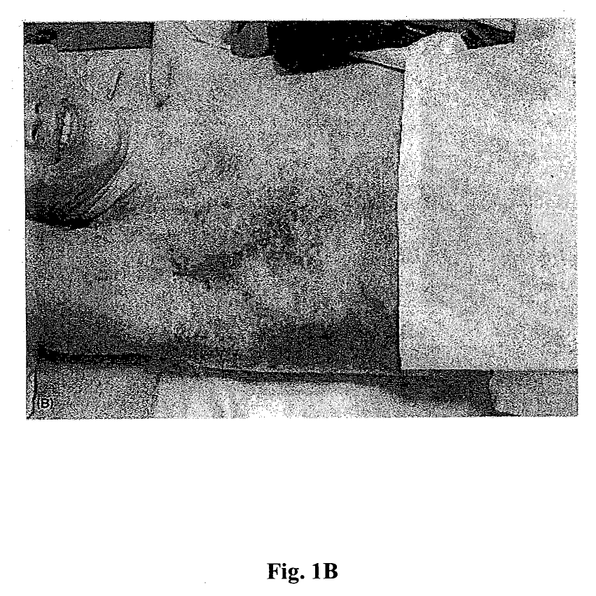

### DETAILED DESCRIPTION OF THE INVENTION

Definitions

The term “effective amount” as used herein refers to an amount sufficient to provide an effect sufficient for the inhibition of the growth of tumor cell in vitro and in vivo.

The term “carrier” of “a pharmaceutically acceptable carrier” as used herein refers to a diluent, an excipient, a recipient and the like for use in preparing admixtures of a pharmaceutical composition.

Treatment efficacy

The use of electrical beam on the biological membrane has been generally known. A technique known as “Eletrochemotherapy.” Electrochemotherapy, involves the application of electron beam to a target cell resulting in the increased permeability of the cell membrane, known as “electroporation,” and allows a greater number of the anticancer drug molecules to enter the target cell's membrane. Thus, a much lower concentration of the drug is introduced without sacrificing efficacy and at the same time reducing or eliminating conventional side effects.

In this present invention, complete remission was achieved for three patients of chest wall skin-infiltrating lesions with one demonstration in the Figure. The cumulative radiation dosages for remission of these chest wall skin lesions were 20, 22 and 28 Gy, respectively. Partial remission was achieved for another three cases with chest wall fungating tumors (Table 1). One patient with chest wall fungating tumor proved resistant to this combined therapy and was classified as a stable disease. The radiation dose for each patient is listed in Table 2. The wound secretion from all of the superficial and fungating wounds was markedly decreased with a change in mean daily needed CD frequency from 4.4 to 0.9 after treatment for 1 month. Relief of chest wall pain assessed by the visual analog scale, VAS, was achieved for all of these seven patients (Table 1).

TABLE 1
Response to treatment

| | Patient | | Change in daily | Reduction |
|---|---|---|---|---|
| | Radiation | Tumor | need of wound | in VAS for |
| Patient | dose (Gy)^{a} | response | CD | skin pain (mm) |
| 1 | 50 | Complete | 6→0 | 75 |
| 2 | 50 | Complete | 4→0 | 65 |
| 3 | 50 | Complete | 4→0 | 80 |
| 4 | 50 | Partial | 5→1 | 70 |
| 5 | 50 | Partial | 3→1 | 65 |
| 6 | 30 | Partial | 5→2 | 75 |
| 7 | 50 | Stable | 4→2 | 55 |

^{a}A dose of 50 Gy was delivered in 25 fractions; 30 Gy in 10 fractions.

Adverse Effects

Table 2 shows the maximal adverse effects developed during and after this combined treatment. The most severe acute radiation dermatitis observed in the study was grade 3 in two patients; however, it had healed satisfactorily 2-3 weeks after completion of the radiation treatment, with none of the patients developing grade 3 chronic radiation dermatitis. No grade 2-4 granulocytosis or leukopenia was noted during the combined therapy. Other adverse reactions included light-headedness during irradiation and moderate fatigue after radiation; however, no changes in performance status were evident. Further, there were no skin pigmentation or significant changes in renal or hepatic function during the course of the combined treatment. Prolongation of the electrocardiogram Q-T interval was not noted.

TABLE 2
Adverse effect and toxicity

| | CTC grade | | | | |
|---|---|---|---|---|---|
| | 0 | 1 | 2 | 3 | 4 |
| Granulocytosis | 6 | 1 | 0 | 0 | 0 |
| Leukopenia | 7 | 0 | 0 | 0 | 0 |
| Thrombocytopenia | 7 | 0 | 0 | 0 | 0 |
| Nausea | 4 | 2 | 1 | 0 | — |
| Anorexia | 4 | 2 | 1 | 0 | 0 |
| Vomit | 6 | 1 | 0 | 0 | 0 |
| Acute radiation dermatitis | 1 | 1 | 3 | 2 | 0 |
| Chronic radiation dermatitis | 4 | 2 | 1 | 0 | 0 |
| Fatigue | 3 | 2 | 2 | 0 | 0 |
| Hyperpigmentation | 7 | 0 | 0 | — | — |

The number shown below grade 0-4 is the case number who developed the adverse effect.

Discussion

In our preliminary investigation of topical As₂O₃ treatment for cutaneous metastatic breast cancer, no significant systemic absorption of As₂0₃ was determined from a pharmacokinetic study [Cheng C F, et al., J Intern Med Taiwan 2003; 14:31-36.]. Additionally, besides the decreased wound secretion and improved local tumor control, other benefits including drying of the skin lesions and reduction in unpleasant odor were also noted.

About 10% of patients with metastatic carcinoma have cutaneous metastases. Management of these metastatic skin lesions with radiation therapy or chemotherapy is usually disappointing. In this study of superficial malignant lesions from breast cancer, we found that combining topical As₂O₃ and radiation therapy not only resulted in satisfactory tumor response, but also achieved a good palliation.

As₂O₃ can arrest the tumor cell cycle at the G2/M phase [Bazarbachi A, et al., Blood 1999; 93:278-283.], with tumor cells most sensitive to radiation at this stage of the cycle [Chakravarthy A, et al., Clin Breast Cancer 2000; 1:68-71.]. This may produce sensitization of the solid tumor cells to radiation [Lew Y S, et al., Cancer Res 2002; 62:4202-4205]. However, there has been no previous suggestion that the combination of As₂O₃ and radiation is useful in the treatment of cutaneous metastatic carcinoma.

A higher clinical response rate in this pilot study was achieved for the combination of topical AS₂O₃ and radiation treatment (42.9% complete remission and 42.9% partial remission) in comparison to topical AS2O₃ alone [Cheng C F, et al., J Intern Med Taiwan 2003; 14:31-36.]. In view of these favorable results, combining topical As₂O₃ and radiation achieves better local control for superficial malignant lesions and further clinical study is recommended.

As As₂O₃ has both apoptosis- and differentiation-inducing activities, development of granulocytosis is a common feature of its clinical use for systemic treatment of leukemia [Soignet S L, et al., N Engl J Med 1998; 339:1341-1348.]. In our study, however, no significant hematological changes were observed. A possible explanation for this finding is that removal of the gel prior to irradiation limited circulatory penetration. Further, although dermatological toxicity such as skin rash has been demonstrated for topical As₂O₃ treatment [Rust D M, & Soignet S L. Oncologist 2001; 6(suppl):32-37.], we did not note any toxicity of this type, perhaps due to the modest duration of daily application (less than 1 h) and treatment (not more than 5 weeks).

With respect to quality of life, the combination of As₂O₃ and radiation treatment appears to offer an effective, tolerable and safe treatment modality for palliative care of breast cancer patients suffering from superficial malignant lesions.

### EXAMPLES

The following examples illustrate methods of preparing, characterizing, and using the composition of the present invention. The examples are in no way intended to limit the scope of the invention.

### Example 1

### Patients and Clinical Protocol

Between December 2001 and March 2003, seven patients of breast cancer with superficial fungating or skin-infiltrating tumors who had undergone standard treatments received topical As₂O₃ and radiotherapy at Mackay Memorial Hospital (Table 3). Eligibility criteria for the study included diagnosis of breast cancer confirmed by pathological examination. In addition, patients had to have relapsed after standard treatments such as surgery, radiation therapy or chemotherapy. Six patients had received post-operative radiation to the chest wall and axillary area. Written informed consent was required, and the study protocol was viewed and approved by the institutional review board of the Mackay Memorial Hospital.

TABLE 3
Patient Characteristics

| | | | | | Pre- |
|---|---|---|---|---|---|
| | | | Cell type of | | radiotherapy |
| Pa- | | | primary | | WHO perfor- |
| tient | Gender | Age | malignancy | Type of lesion | mance status |
| 1 | Female | 53 | IDC | Skin infiltration | 1 |
| 2 | Female | 46 | IDC | Skin infiltration | 2 |
| 3 | Female | 62 | LIC | Skin infiltration | 2 |
| 4 | Female | 33 | IDC | Fungating tumor | 2 |
| 5 | Female | 41 | LIC | Fungating tumor | 2 |
| 6 | Female | 57 | IDC | Fungating tumor | 3 |
| 7 | Female | 71 | IDC | Fungating tumor | 3 |

IDC: infiltrating ductal carcinoma;
LIC: lobular invasive carcinoma.

### Example 2

### Preparation of As₂O₃ Gel Pharmaceutical

The pharmaceutical composition per unit dose in the gel consists of:

| Ingredient | Content (mg) | Percentage (%) |
|---|---|---|
| As₂O₃ | 0.5 | 0.05 |
| Carbomer 940 | 95 | 0.95 |
| PEG400 | 90 | 9 |
| Propyl paraben | 100 | 10 |
| Purified water | 800 | 80 |
| Total | 1000 | 100 |

### Example 3

### Treatment Program

As₂O₃ gel prepared by Example 2 was administered in daily topical doses that ranged from 0.01 to 0.5 mg/cm2/day, preferred from 0.05-0.15 mg/cm2/day. The gel was administered 1 h prior to the daily radiation treatment and was subsequently removed 5 min prior to the radiation exposure. Electron beam radiotherapy was delivered using a linear accelerator (9-12 MeV, dose rate 2.4 Gy/min; Clinac 1800; Varian Associates, Palo Alto, Calif.). Radiation was administered 5 days a week (total dose: 50 Gy in 25 fractions or 30 Gy in 10 fractions).

### Example 4

### Assessment of Response

Observation for tumor response and skin reaction was conducted daily, with photographs taken weekly. A complete response was defined as the disappearance of all known skin lesions within radiation fields confirmed by subsequent observation at least 4 weeks apart. A partial response was defined as a decrease in the sum of the maximal perpendicular diameters for involved skin area within radiation fields by more than 50% which was confirmed by subsequent observation at least 4 weeks apart. Progressive disease was defined as at least 25% increase in the sum of the maximal perpendicular diameters for involved skin area or the appearance of new lesions within radiation fields. All other tumor outcomes were classified as stable disease. For efficacy of pain control, chest wall pain was estimated using a visual analog scale (VAS) as a subjective assessment. The VAS scores before treatment were set as the baseline for every patient, and we compared the changes between subsequent data after treatment and the baseline data. When differences were greater than 50 mm in a 100-mm scale, we identified this patient as a responder. The daily need for changing dressings (CD) of wounds was also recorded by nurses in charge of each patient. The result is shown in Table 1.

### Example 5

### Monitoring for Adverse Effects

With regard to drug absorption via skin, possible systemic adverse effects were evaluated. Serial blood counts and serum chemistry profiles were performed. Serum chemistry profiles, including alanine aminotransferase, aspartate aminotransferase, blood urine nitrogen and creatinine, were measured using a Synchron LX20 spectrophotometer (Beckman Coulter, San Diego, Calif.). An electrocardiogram was recorded before and after irradiation for each patient. Adverse effect and toxicity were evaluated according to the Common Toxicity Criteria (CTC) version 2.0 published by the DCTD, NCI, NIH and DHHS in 1999. The result is shown in Table 2.

Although the invention has been described with respect to particular embodiments, it will be apparent to those skilled in the art that various changes and modifications can be made without departing from the invention.

### REFERENCES

1. Soignet S L, Maslak P, Wang Z G, et al. Complete remission after treatment of acute promyelocytic leukemia with arsenic trioxide. N Engl J Med 1998; 339:1341-1348.

2. Bazarbachi A, Hermine O. Treatment of adult T-cell leukaemia/lymphoma: current strategy and future perspectives. Virus Res 2001; 78:79-92.

3. Shao W, Fanelli M, Ferrara F F, et al. Arsenic trioxide as an inducer of apoptosis and loss of PML/RAR alpha protein in acute promyelocytic leukemia cells. J Natl Cancer I 1998; 90:124-133.

4. Siu K P, Chan J Y, Fung K P. Effect of arsenic trioxide on human hepatocellular carcinoma HepG2 cells: inhibition of proliferation and induction of apoptosis. Life Sci 2002; 71:275-285.

5. Li X, Ding X, Adrian T E. Arsenic trioxide inhibits proliferation and induces apoptosis in pancreatic cancer cells. Anticancer Res 2002; 22:2205-2213.

6. Jiang X H, Wong B C, Yuen S T, et al. Arsenic trioxide induces apoptosis in human gastric cancer cells through up-regulation of p53 and activation of caspase-3. Int J Cancer 2001; 91:173-179.

7. Wang Z. Y., Arsenic compounds as anticancer agents. Cancer Chemother Pharmacol (2001), 48 (suppl 1): S72-S76

8. Murgo A. J., Clinical trials of arsenic trioxide in hematologic and solid tumors: overview of the National Cancer Institute Cooperative Research and Development Studies. The Oncologist (2001), 6 (suppl 2):22-28

9. Chun Y J, Park I C, Park M J, et al. Enhancement of radiation response in human cervical cancer cells in vitro and in vivo by arsenic trioxide (AS2O₃). FEBS Left 2002; 519:195-200.

10. Cheng C F, Hsu H H, Huang M L, Huang M J. Study on the systemic absorption of topically applied AS₂O₃ lotion in patients with cutaneous metastatic breast cancer—report of 2 cases. J Intern Med Taiwan 2003; 14:31-36.

11. Bazarbachi A, El Sabban M E, Nasr R, et al. Arsenic trioxide and interferonalpha synergize to induce cell cycle arrest and apoptosis in human T-cell lymphotropic virus type I-transformed cells. Blood 1999; 93:278-283.

12. Chakravarthy A, Nicholson B, Kelley M, et al. A pilot study of neoadjuvant paclitaxel and radiation with correlative molecular studies in stage II/III breast cancer. Clin Breast Cancer 2000; 1:68-71.

13. Lew Y S, Kolozsvary A, Brown S L, Kim J H. Synergistic interaction with arsenic trioxide and fractionated radiation in locally advanced murine tumor. Cancer Res 2002; 62:4202-4205.

14. Rust D M, Soignet S L. Risk/benefit profile of arsenic trioxide. Oncologist 2001; 6(suppl):32-37.

1. A method for treating a human patient having a cutaneuous metastatic cancer comprising:
(a) administering topically an arsenic-containing pharmaceutical composition to a site of said cutaneuous metastatic cancer of human patient, said arsenic-containing pharmaceutical composition including a therapeutically effective amount of an arsenic-containing compound and a pharmaceutically acceptable carrier; and (b) applying transcutaneously an electron beam to the site.

2. The method according to claim 1, further comprising the step of removing said arsenic-containing pharmaceutical composition from the site prior to step (b).

3. The method according to claim 1 wherein said cutaneuous metastatic cancer is selected from the group consisting of breast cancer, colon cancer, ovary cancer, lung cancer, head and neck squamous cancer, and oral cavity cancer.

4. The method according to claim 1, wherein said cutaneuous metastatic cancer is cutaneuous metastatic breast cancer.

5. The method according to claim 1, wherein said arsenic-containing compound is selected from the group consisting of AS₂O₃, As₂S₃, As₂S₂ and a combination thereof.

6. The method according to claim 5, wherein the arsenic-containing compound is As₂O₃.

7. The method according to claim 1, wherein said arsenic-containing pharmaceutical composition is an arsenic-containing gel formulation.

8. The method according to claim 7, wherein said arsenic-containing pharmaceutical composition is administered in a daily dose ranging from 0.01 to 0.5 mg/cm².

9. The method according to claim 8, wherein said daily dose ranges from 0.05 to 0.15 mg/cm².

10. The method according to claim 1, wherein said electron beam is applied in a total radiation dose ranging from 30 to 50 Gy/5 days.
